SEQUENCE LISTING (1 sequence)

SEQ ID NO: 1 - protein, 5 aa, synthetic construct
  REGION 1..5: note = exemplary peptide
KKKKK

(19) United States
(12) Patent Application Publication
(10) Pub. No.: US 2025/0049902 A1
(43) Pub. Date: Feb. 13, 2025

(54) Method of Developing a Peptide-Based Vaccine Conjugated with 1V209

(71) Applicant: VERSITECH LIMITED, Hong Kong (CN)

(72) Inventors: Jiandong HUANG, Hong Kong (CN); Yefan HU, Hong Kong (CN)

(73) Assignee: VERSITECH LIMITED, Hong Kong (CN)

(21) Appl. No.: 18/721,343

(22) PCT Filed: Dec. 6, 2022
(86) PCT No.: PCT/CN2022/136832

§ 371 Date: Jun. 18, 2024

Related U.S. Application Data
(60) Provisional application No. 63/265,780, filed on Dec. 20, 2021.

(51) Int. Cl. A61K 39/00; A61K 39/385

(52) U.S. Cl. CPC A61K 39/0011; A61K 39/385

(57) ABSTRACT

Provided are a composition comprising a peptide conjugated to a TLR agonist, including TLR7 agonist 1v209, and methods of using the composition. The methods comprise administering the peptide-based vaccine conjugate to a subject. The peptide-based vaccine conjugate inhibits tumor growth and induces antigen-specific B and T cell responses in a subject.

### BACKGROUND OF THE INVENTION

Peptide-based vaccines have been widely used in cancer vaccines in clinical trial [1]. Due to the relatively small size, peptides cannot easily induce immune responses by themselves. Therefore, peptide-based vaccines need to be injected with adjuvants to optimize the efficacy. Currently, peptides are simply mixed with adjuvants, like poly(I.C) [1] or TLR7 agonist [2]. It was reported that 1V209, chemically named 4-[[6-Amino-7,8-dihydro-2-(2-methoxyethoxy)-8-oxo-9H-purin-9-yl]methyl]-benzoic acid with the CAS Number of 1062444-54-5, can stimulate the TLR7 pathway as an agonist [3], which showed the potential to be an adjuvant in treating cancer [4]. However, 1V209, as a small molecule, can hardly induce antigen specific immune responses when simply mixed with antigens [3]. Past studies showed the conjugation of adjuvants and antigens can increase the immune response [5].

Therefore, a novel conjugated peptide is needed stimulate antigen-specific immune responses in treating cancer.

### BRIEF SUMMARY OF THE INVENTION

The subject invention pertains to compositions and methods of treating cancer. In certain embodiments, the compositions comprise a peptide-based vaccine conjugate. In certain embodiments, the compositions comprise a novel 1V209-conjugated peptide that can stimulate antigen-specific immune responses in treating cancer. In certain embodiments, both B cell and T cell antigen-specific immune responses can be stimulated.

### BRIEF DESCRIPTION OF THE DRAWINGS

FIG. 1 The 1V209-conjugated peptide structure. A. The structure of 1V209 (CAS No. 1062444-54-5). B. The structure of the antigen. C. The 1V209-conjugated peptide structure.

FIGS. 2A-2E Immunogenicity test of peptide-based self-adjuvanting vaccine. FIG. 2A. Administration process in mouse model for immunogenicity test, as well as dose of different groups. FIG. 2B. IFN-γ ELISpot results of 1V209-conjugated peptide vaccine (conjugates) comparing with Imiquimod or poly(I:C)-adjuvanting original peptide. Here, students' t test is used. * P<0.05, ** P<0.01. FIG. 2C. The percentage of IFN-γ⁺ T cells in CD8⁺ or CD4⁺ T cells measured by flow cytometry. FIG. 2D. CD8⁺ IFN-γ⁺ T cell flow cytometry result. FIG. 2E. CD4⁺ IFN-γ⁺ T cell flow cytometry result.

FIGS. 3A-3E Peptide-based self-adjuvanting vaccine in cancer treatment. FIG. 3A. The process of tumour inoculation and treatment by 1V209-conjugated peptide (conjugates) or peptide adjuvanting with poly(I:C). 100 μg of each neoantigen were immunized at Day 0, 3, 7, and 14. At Day 17, the mice were sacrificed for ELISpot assay. FIG. 3B. Tumour growth kinetics of control and conjugates or peptide+poly(I:C) treatment groups. FIG. 3C. Tumour sizes of different treatment groups at Day 17. Here, students' t test is used. * P<0.05, ** P<0.01. FIG. 3D. The optical density (OD) neoantigen-specific IgG at 450 nm. 1:50 mouse sera were used. FIG. 3E. IFN-γ ELISpot results of 1V209-conjugated peptide (conjugates) comparing with poly(I:C)-adjuvanting original peptide. Here, students' t test is used. * P<0.05; ** P<0.01; ns, no significance.

### BRIEF DESCRIPTION OF THE SEQUENCES

SEQ ID NO: 1 Exemplary peptide

### DETAILED DISCLOSURE OF THE INVENTION

### Selected Definitions

As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising”. The transitional terms/phrases (and any grammatical variations thereof) “comprising”, “comprises”, “comprise”, “consisting essentially of”, “consists essentially of”, “consisting” and “consists” can be used interchangeably.

The phrases “consisting essentially of” or “consists essentially of” indicate that the claim encompasses embodiments containing the specified materials or steps and those that do not materially affect the basic and novel characteristic(s) of the claim.

The term “about” means within an acceptable error range for the particular value as determined by one of ordinary skill in the art, which depends in part on how the value is measured, i.e., the limitations of the measurement system. In the context of compositions containing amounts of ingredients where the term “about” is used, these compositions contain the stated amount of the ingredient with a variation (error range) of 0-10% around the value (X±10%). In other contexts, the term “about” provides a variation (error range) of 0-10% around a given value (X±10%). As is apparent, this variation represents a range that is up to 10% above or below a given value, for example, X±1%, X±2%, X±3%, X±4%, X±5%, X±6%, X 7%, X±8%, X±9%, or X±10%.

In the present disclosure, ranges are stated in shorthand to avoid having to set out at length and describe each and every value within the range. Any appropriate value within the range can be selected, where appropriate, as the upper value, lower value, or the terminus of the range. For example, a range of 0.1-1.0 represents the terminal values of 0.1 and 1.0, as well as the intermediate values of 0.2, 0.3, 0.4, 0.5, 0.6, 0.7, 0.8, 0.9, and all intermediate ranges encompassed within 0.1-1.0, such as 0.2-0.5, 0.2-0.8, 0.7-1.0, etc. Values having at least two significant digits within a range are envisioned, for example, a range of 5-10 indicates all the values between 5.0 and 10.0 as well as between 5.00 and 10.00 including the terminal values. When ranges are used herein, combinations and subcombinations of ranges (e.g., subranges within the disclosed range) and specific embodiments therein are explicitly included.

As used herein, the term “subject” refers to an animal, needing or desiring delivery of the benefits provided by a therapeutic composition. The animal may be for example, humans, pigs, horses, goats, cats, mice, rats, dogs, apes, fish, chimpanzees, orangutans, guinea pigs, hamsters, cows, sheep, birds, chickens, as well as any other vertebrate or invertebrate. These benefits can include, but are not limited to, the treatment of a health condition, disease or disorder; prevention of a health condition, disease or disorder; immune health; enhancement of the function of enamel, an organ, tissue, or system in the body. The preferred subject in the context of this invention is a human. The subject can be of any age or stage of development, including infant, toddler, adolescent, teenager, adult, or senior.

As used herein, the terms “therapeutically-effective amount,” “therapeutically-effective dose,” “effective amount,” and “effective dose” are used to refer to an amount or dose of an peptide-based vaccine conjugate or composition that, when administered to a subject, is capable of treating or improving a condition, disease, or disorder in a subject or that is capable of providing enhancement in health or function to an organ, tissue, or body system. In other words, when administered to a subject, the amount is “therapeutically effective.” The actual amount will vary depending on a number of factors including, but not limited to, the particular condition, disease, or disorder being treated or improved; the severity of the condition; the particular organ, tissue, or body system of which enhancement in health or function is desired; the weight, height, age, and health of the patient; and the route of administration.

As used herein, the term “treatment” refers to eradicating, reducing, ameliorating, or reversing a sign or symptom of a health condition, disease or disorder to any extent, and includes, but does not require, a complete cure of the condition, disease, or disorder. Treating can be curing, improving, or partially ameliorating a disorder. “Treatment” can also include improving or enhancing a condition or characteristic, for example, bringing the function of a particular system in the body to a heightened state of health or homeostasis.

As used herein, “preventing” a health condition, disease, or disorder refers to avoiding, delaying, forestalling, or minimizing the onset of a particular sign or symptom of the condition, disease, or disorder. Prevention can, but is not required, to be absolute or complete; meaning, the sign or symptom may still develop at a later time. Prevention can include reducing the severity of the onset of such a condition, disease, or disorder, and/or inhibiting the progression of the condition, disease, or disorder to a more severe condition, disease, or disorder.

By “reduces” is meant a negative alteration of at least 1%, 5%, 10%, 25%, 50%, 75%, or 100%.

By “increases” is meant as a positive alteration of at least 1%, 5%, 10%, 25%, 50%, 75%, or 100%.

In some embodiments of the invention, the method comprises administration of multiple doses of the compositions of the subject invention. The method may comprise administration of therapeutically effective doses of a composition comprising the peptide-based vaccine conjugates of the subject invention as described herein three times a week, once a week, or more frequency. In some embodiments, doses are administered over the course of 1 week, 2 weeks, or more than 3 weeks. Moreover, treatment of a subject with a therapeutically effective amount of the compositions of the invention can include a single treatment or can include a series of treatments. It will also be appreciated that the effective dosage of a peptide-based vaccine conjugate used for treatment may increase or decrease over the course of a particular treatment. Changes in dosage may result and become apparent from the results of diagnostic assays or imaging techniques for detecting tumor sizes known in the art. In some embodiments of the invention, the method comprises administration of the compositions at several time per day, including but not limiting to 2 times per day, 3 times per day, and 4 times per day.

The terms “antagonist” and “inhibitor” may be used interchangeably, and they refer to a compound having the ability to inhibit a biological function of a target protein, whether by inhibiting the activity or expression of the target protein. Accordingly, the terms “antagonist” and “inhibitor” are defined in the context of the biological role of the target protein.

The terms “agonists” and “activators” and their synonyms may be used interchangeably, and they refer to a compound having the ability to activate a biological function of a target protein, whether by increasing the activity or expression of the target protein. Accordingly, the terms “agonist” and “activator” are defined in the context of the biological role of the target protein.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Peptide-based Vaccine Conjugated with 1V209 and Methods of Use

The present invention describes a peptide-based vaccine conjugate and methods of making and using said peptide based-vaccine conjugate. In certain embodiments, a peptide can be conjugated to a Toll-like receptor agonist, including, for example, a Toll-like receptor 7 agonist (TLR7a). In certain embodiments, the TLR7a can be 1V209. In certain embodiments, the peptide conjugated to the Toll-like receptor agonist can comprise at least 5, 6, 7, 8, 9, 10, or more amino acid residues. In certain embodiments, the amino acid residues of the peptide comprise at least 1, 2, 3, 4, 5 or more amino acid residue conjugated with 1V209. In certain embodiments, an amino acid residue can be lysine. In certain embodiments, the peptide comprises the sequence KKKKK (SEQ ID NO:1). In certain embodiments, a Toll-like receptor agonist can be conjugated to each amino acid residue of the peptide.

In preferred embodiments, the compositions and methods according to the subject invention utilize a peptide-based vaccine conjugate, such as, for example, a peptide-based vaccine conjugate according to formula (I). Peptide-based vaccine conjugate compounds may be added to compositions at concentrations of 0.01 to 90% by weight (wt %), preferably 0.1 to 50 wt %, and more preferably 0.1 to 20 wt %. In another embodiment, a purified peptide-based vaccine conjugate compound may be in combination with an acceptable carrier and/or excipient, in that peptide-based vaccine conjugate compound may be presented at concentrations of 0.001 to 50% (v/v), preferably, 0.01 to 20% (v/v), more preferably, 0.02 to 10% (v/v). In certain embodiments, the peptide-based vaccine conjugate can be administered to a subject at a dosage of about 3-50 mg/kg.

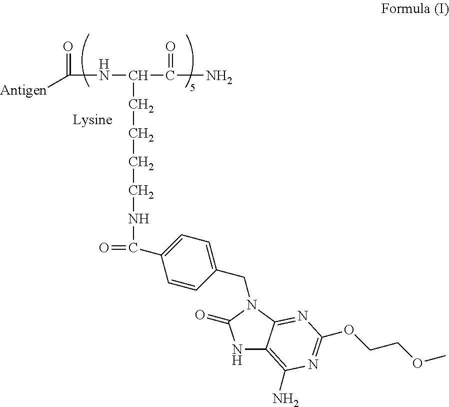

In certain embodiments, the peptide, comprising, for example, lysine residues, can be conjugated with the TLR agonist molecules, such as, for example, 1V209, by forming amide bonds between amino groups on side chain of the lysine residue and carboxyl groups of TLR agonist. In certain embodiments, the peptide-based vaccine conjugate can induce immune responses, such as, for example, T cell or B cell responses, after administration into subjects. In certain embodiments, the peptide-based vaccine conjugate can be administered to inhibit tumor cells because the administration of tumor specific antigens generated from single nucleotide variations (SNVs), insertions/deletions (Indels), or other types of DNA variations, which change the coding sequences of normal genes, can induce tumor-specific immune responses, such as, for example, T cell or B cell responses.

In certain embodiments, a synthetic peptide vaccine can be used to stimulate T cell responses against specific antigens to treat cancer. Such peptide vaccines can be administrated with vaccine adjuvants, such as, for example, TLR7a. 1V209 can stimulate TLR7 pathway as an agonist, which showed the potential to be an adjuvant in vaccine. However, 1V209 alone, as a small molecule, can hardly induce specific immune responses when simply mixed with antigens.

In certain embodiments, the peptide-based vaccine conjugates can induce a T cell response. In certain embodiments, the peptide-based vaccine conjugates can induce a B cell response in terms of an antigen-specific antibody. In certain embodiments, the peptide-based vaccine conjugates can induce antigen specific CD4⁺ and CD8⁺ T cell responses. In certain embodiments, a peptide-based conjugated vaccine can induce both antigen specific-B cell responses and antigen specific CD4⁺ and CD8⁺ T cell responses. Antigen-specific B cells can release antibodies targeting tumors or help T cell to kill tumor cells. Antigen-specific T cells can recognize tumor cells with such antigens, and then release granzyme B, cytokines and chemokines to induce tumor cell killing.

Cancers suitable for treatment according to the disclosed methods include, but are not limited to: Acanthoma, Acinic cell carcinoma, Acoustic neuroma, Acral lentiginous melanoma, Acrospiroma, Acute eosinophilic leukemia, Acute lymphoblastic leukemia, Acute megakaryoblastic leukemia, Acute monocytic leukemia, Acute myeloblastic leukemia with maturation, Acute myeloid dendritic cell leukemia, Acute myeloid leukemia, Acute promyelocytic leukemia, Adamantinoma, Adenocarcinoma, Adenoid cystic carcinoma, Adenoma, Adenomatoid odontogenic tumor, Adrenocortical carcinoma, Adult T-cell leukemia, Aggressive NK-cell leukemia, AIDS-related cancers, AIDS-related lymphoma, Alveolar soft part sarcoma, Ameloblastic fibroma, Anal cancer, Anaplastic large cell lymphoma, Anaplastic thyroid cancer, Angioimmunoblastic T-cell lymphoma, Angiomyolipoma, Angiosarcoma, Appendix cancer, Astrocytoma, Atypical teratoid rhabdoid tumor, Basal cell carcinoma, Basal-like carcinoma, B-cell leukemia, B-cell lymphoma, Bellini duct carcinoma, Biliary tract cancer, Bladder cancer, Blastoma, Bone cancer, Bone tumor, Breast cancer, Brenner tumor, Bronchial tumor, Bronchioloalveolar carcinoma, Brown tumor, Burkitt's lymphoma, Cancer of unknown primary site, Carcinoid tumor, Carcinoma, Carcinoma in situ, Carcinoma of the penis, Carcinoma of unknown primary site, Carcinosarcoma, Castleman disease, Central nervous system embryonal tumor, Cerebellar astrocytoma, Cerebral astrocytoma, Cervical cancer, Cholangiocarcinoma, Chondroma, Chondrosarcoma, Chordoma, Choriocarcinoma, Choroid plexus papilloma, Chronic lymphocytic leukemia, Chronic monocytic leukemia, Chronic myelogenous leukemia, Chronic myeloproliferative disorder, Chronic neutrophilic leukemia, Clear-cell tumor, Colon cancer, Colorectal cancer, Craniopharyngioma, Cutaneous T-cell lymphoma, Degos disease, Dermatofibrosarcoma protuberans, Dermoid cyst, Desmoplastic small round cell tumor, Diffuse large B cell lymphoma, Dysembryoplastic neuroepithelial tumor, Embryonal carcinoma, Endodermal sinus tumor, Endometrial cancer, Endometrial uterine cancer, Endometrioid tumor, Enteropathy-associated T-cell lymphoma, Ependymoblastoma, Ependymoma, Epithelioid sarcoma, Erythroleukemia, Esophageal cancer, Esthesioneuroblastoma, Ewing family of tumors, Ewing sarcoma, Extracranial germ cell tumor, Extragonadal germ cell tumor, Extrahepatic bile duct cancer, Extramammary Paget's disease, Fallopian tube cancer, Fetus in fetu, Fibroma, Fibrosarcoma, Follicular lymphoma, Follicular thyroid cancer, Gallbladder cancer, Ganglioglioma, Ganglioneuroma, Gastric cancer, Gastric lymphoma, Gastrointestinal cancer, Gastrointestinal carcinoid tumor, Gastrointestinal stromal tumor, Germ cell tumor, Germinoma, Gestational choriocarcinoma, Gestational trophoblastic tumor, Giant cell tumor of bone, Glioblastoma multiforme, Glioma, Gliomatosis cerebri, Glomus tumor, Glucagonoma, Gonadoblastoma, Granulosa cell tumor, Hairy cell leukemia, Head and neck cancer, Heart cancer, Hemangioblastoma, Hemangiopericytoma, Hemangiosarcoma, Hematological malignancy, Hepatocellular carcinoma, Hepatosplenic T-cell lymphoma, Hereditary breast-ovarian cancer syndrome, Hodgkin's lymphoma, Hypopharyngeal cancer, Hypothalamic glioma, Inflammatory breast cancer, Intraocular melanoma, Islet cell carcinoma, Islet cell tumor, Juvenile myelomonocytic leukemia, Kaposi's sarcoma, Kidney cancer, Klatskin tumor, Krukenberg tumor, Laryngeal cancer, Lentigo maligna melanoma, Leukemia, Lip and oral cavity cancer, Liposarcoma, Lung cancer, Luteoma, Lymphangioma, Lymphangiosarcoma, Lymphoepithelioma, Lymphoid leukemia, Lymphoma, Macroglobulinemia, Malignant fibrous histiocytoma, Malignant fibrous histiocytoma of bone, Malignant glioma, Malignant mesothelioma, Malignant peripheral nerve sheath tumor, Malignant rhabdoid tumor, Malignant triton tumor, MALT lymphoma, Mantle cell lymphoma, Mast cell leukemia, Mediastinal germ cell tumor, Mediastinal tumor, Medullary thyroid cancer, Medulloblastoma, Medulloepithelioma, Melanoma, Meningioma, Merkel cell carcinoma, Mesothelioma, Metastatic squamous neck cancer with occult primary, Metastatic urothelial carcinoma, Mixed Millerian tumor, Monocytic leukemia, Mouth cancer, Mucinous tumor, Multiple endocrine neoplasia syndrome, Multiple myeloma, Mycosis fungoides, Myelodysplasia disease, Myelodysplasia syndromes, Myeloid leukemia, Myeloid sarcoma, Myeloproliferative disease, Myxoma, Nasal cavity cancer, Nasopharyngeal cancer, Nasopharyngeal carcinoma, Neoplasm, Neurinoma, Neuroblastoma, Neurofibroma, Neuroma, Nodular melanoma, Non-Hodgkin's lymphoma, Nonmelanoma skin cancer, Non-small cell lung cancer, Ocular oncology, Oligoastrocytoma, Oligodendroglioma, Oncocytoma, Optic nerve sheath meningioma, Oral cancer, Oropharyngeal cancer, Osteosarcoma, Ovarian cancer, Ovarian epithelial cancer, Ovarian germ cell tumor, Ovarian low malignant potential tumor, Paget's disease of the breast, Pancoast tumor, Pancreatic cancer, Papillary thyroid cancer, Papillomatosis, Paraganglioma, Paranasal sinus cancer, Parathyroid cancer, Penile cancer, Perivascular epithelioid cell tumor, Pharyngeal cancer, Pheochromocytoma, Pineal parenchymal tumor of intermediate differentiation, Pineoblastoma, Pituicytoma, Pituitary adenoma, Pituitary tumor, Plasma cell neoplasm, Pleuropulmonary blastoma, Polyembryoma, precursor T-lymphoblastic lymphoma, Primary central nervous system lymphoma, Primary effusion lymphoma, Primary hepatocellular cancer, Primary liver cancer, Primary peritoneal cancer, Primitive neuroectodermal tumor, Prostate cancer, Pseudomyxoma peritonei, Rectal cancer, Renal cell carcinoma, Respiratory tract carcinoma involving the NUT gene on chromosome 15, Retinoblastoma, Rhabdomyoma, Rhabdomyosarcoma, Richter's transformation, Sacrococcygeal teratoma, Salivary gland cancer, Sarcoma, Schwannomatosis, Sebaceous gland carcinoma, Secondary neoplasm, Seminoma, Serous tumor, Sertoli-Leydig cell tumor, Sex cord-stromal tumor, Sézary syndrome, Signet ring cell carcinoma, Skin cancer, Small blue round cell tumor, Small cell carcinoma, Small cell lung cancer, Small cell lymphoma, Small intestine cancer, Soft tissue sarcoma, Somatostatinoma, Soot wart, Spinal cord tumor, Spinal tumor, Splenic marginal zone lymphoma, Squamous cell carcinoma, Stomach cancer, Superficial spreading melanoma, Supratentorial primitive neuroectodermal tumor, Surface epithelial-stromal tumor, Synovial sarcoma, T-cell acute lymphoblastic leukemia, T-cell large granular lymphocyte leukemia, T-cell leukemia, T-cell lymphoma, T-cell prolymphocytic leukemia, Teratoma, Terminal lymphatic cancer, Testicular cancer, Thecoma, Throat cancer, Thymic carcinoma, Thymoma, Thyroid cancer, Transitional cell cancer of renal pelvis and ureter, Transitional cell carcinoma, Urachal cancer, Urethral cancer, Urogenital neoplasm, Uterine sarcoma, Uveal melanoma, Vaginal cancer, Verner-Morrison syndrome, Verrucous carcinoma, Visual pathway glioma, Vulvar cancer, Waldenstrom macroglobulinemia, Warthin's tumor, Wilms' tumor, or any combinations thereof. In preferred embodiments, the compositions can be used to treat bladder cancer or liver cancer.

In one embodiment, the subject compositions are formulated as an orally-consumable product, such as, for example a food item, capsule, pill, or drinkable liquid. An orally deliverable pharmaceutical is any physiologically active substance delivered via initial absorption in the gastrointestinal tract or into the mucus membranes of the mouth. The topic compositions can also be formulated as a solution that can be administered via, for example, injection, which includes intravenously, intraperitoneally, intramuscularly, intrathecally, or subcutaneously. In other embodiments, the subject compositions are formulated to be administered via the skin through a patch or directly onto the skin for local or systemic effects. The compositions can be administered sublingually, buccally, rectally, or vaginally. Furthermore, the compositions can be sprayed into the nose for absorption through the nasal membrane, nebulized, inhaled via the mouth or nose, or administered in the eye or ear.

Orally consumable products according to the invention are any preparations or compositions suitable for consumption, for nutrition, for oral hygiene, or for pleasure, and are products intended to be introduced into the human or animal oral cavity, to remain there for a certain period of time, and then either be swallowed (e.g., food ready for consumption or pills) or to be removed from the oral cavity again (e.g., chewing gums or products of oral hygiene or medical mouth washes). While an orally-deliverable pharmaceutical can be formulated into an orally consumable product, and an orally consumable product can comprise an orally deliverable pharmaceutical, the two terms are not meant to be used interchangeably herein.

Orally consumable products include all substances or products intended to be ingested by humans or animals in a processed, semi-processed, or unprocessed state. This also includes substances that are added to orally consumable products (particularly food and pharmaceutical products) during their production, treatment, or processing and intended to be introduced into the human or animal oral cavity.

Orally consumable products can also include substances intended to be swallowed by humans or animals and then digested in an unmodified, prepared, or processed state; the orally consumable products according to the invention therefore also include casings, coatings, or other encapsulations that are intended to be swallowed together with the product or for which swallowing is to be anticipated.

In one embodiment, the orally consumable product is a capsule, pill, syrup, emulsion, or liquid suspension containing a desired orally deliverable substance. In one embodiment, the orally consumable product can comprise an orally deliverable substance in powder form, which can be mixed with water or another liquid to produce a drinkable orally-consumable product.

In some embodiments, the orally-consumable product according to the invention can comprise one or more formulations intended for nutrition or pleasure. These particularly include baking products (e.g., bread, dry biscuits, cake, and other pastries), sweets (e.g., chocolates, chocolate bar products, other bar products, fruit gum, coated tablets, hard caramels, toffees and caramels, and chewing gum), alcoholic or non-alcoholic beverages (e.g., cocoa, coffee, green tea, black tea, black or green tea beverages enriched with extracts of green or black tea, Rooibos tea, other herbal teas, fruit-containing lemonades, isotonic beverages, soft drinks, nectars, fruit and vegetable juices, and fruit or vegetable juice preparations), instant beverages (e.g., instant cocoa beverages, instant tea beverages, and instant coffee beverages), meat products (e.g., ham, fresh or raw sausage preparations, and seasoned or marinated fresh meat or salted meat products), eggs or egg products (e.g., dried whole egg, egg white, and egg yolk), cereal products (e.g., breakfast cereals, muesli bars, and pre-cooked instant rice products), dairy products (e.g., whole fat or fat reduced or fat-free milk beverages, rice pudding, yoghurt, kefir, cream cheese, soft cheese, hard cheese, dried milk powder, whey, butter, buttermilk, and partly or wholly hydrolyzed products containing milk proteins), products from soy protein or other soy bean fractions (e.g., soy milk and products prepared thereof, beverages containing isolated or enzymatically treated soy protein, soy flour containing beverages, preparations containing soy lecithin, fermented products such as tofu or tempeh products prepared thereof and mixtures with fruit preparations and, optionally, flavoring substances), fruit preparations (e.g., jams, fruit ice cream, fruit sauces, and fruit fillings), vegetable preparations (e.g., ketchup, sauces, dried vegetables, deep-freeze vegetables, pre-cooked vegetables, and boiled vegetables), snack articles (e.g., baked or fried potato chips (crisps) or potato dough products and extrudates on the basis of maize or peanuts), products on the basis of fat and oil or emulsions thereof (e.g., mayonnaise, remoulade, and dressings), other ready-made meals and soups (e.g., dry soups, instant soups, and pre-cooked soups), seasonings (e.g., sprinkle-on seasonings), sweetener compositions (e.g., tablets, sachets, and other preparations for sweetening or whitening beverages or other food). The present compositions may also serve as semi-finished products for the production of other compositions intended for nutrition or pleasure.

The subject composition can further comprise one or more pharmaceutically acceptable carriers, and/or excipients, and can be formulated into preparations, for example, solid, semi-solid, liquid, or gaseous forms, such as tablets, capsules, powders, granules, ointments, solutions, suppositories, injections, inhalants, and aerosols.

The term “pharmaceutically acceptable” as used herein means compatible with the other ingredients of a pharmaceutical composition and not deleterious to the recipient thereof.

Carriers and/or excipients according the subject invention can include any and all solvents, diluents, buffers (such as, e.g., neutral buffered saline, phosphate buffered saline, or optionally Tris-HCl, acetate or phosphate buffers), oil-in-water or water-in-oil emulsions, aqueous compositions with or without inclusion of organic co-solvents suitable for, e.g., IV use, solubilizers (e.g., Polysorbate 65, Polysorbate 80), colloids, dispersion media, vehicles, fillers, chelating agents (e.g., EDTA or glutathione), amino acids (e.g., glycine), proteins, disintegrants, binders, lubricants, wetting agents, emulsifiers, sweeteners, colorants, flavorings, aromatizers, thickeners (e.g. carbomer, gelatin, or sodium alginate), coatings, preservatives (e.g., Thimerosal, benzyl alcohol, polyquaterium), antioxidants (e.g., ascorbic acid, sodium metabisulfite), tonicity controlling agents, absorption delaying agents, adjuvants, bulking agents (e.g., lactose, mannitol) and the like. The use of carriers and/or excipients in the field of drugs and supplements is well known. Except for any conventional media or agent that is incompatible with the target health-promoting substance or with the composition, carrier or excipient use in the subject compositions may be contemplated.

In one embodiment, the compositions of the subject invention can be made into aerosol formulations so that, for example, it can be nebulized or inhaled. Suitable pharmaceutical formulations for administration in the form of aerosols or sprays are, for example, powders, particles, solutions, suspensions or emulsions. Formulations for oral or nasal aerosol or inhalation administration may also be formulated with carriers, including, for example, saline, polyethylene glycol or glycols, DPPC, methylcellulose, or in mixture with powdered dispersing agents or fluorocarbons. Aerosol formulations can be placed into pressurized propellants, such as dichlorodifluoromethane, propane, nitrogen, fluorocarbons, and/or other solubilizing or dispersing agents known in the art. Illustratively, delivery may be by use of a single-use delivery device, a mist nebulizer, a breath-activated powder inhaler, an aerosol metered-dose inhaler (MDI), or any other of the numerous nebulizer delivery devices available in the art. Additionally, mist tents or direct administration through endotracheal tubes may also be used.

In one embodiment, the compositions of the subject invention can be formulated for administration via injection, for example, as a solution or suspension. The solution or suspension can comprise suitable non-toxic, parenterally-acceptable diluents or solvents, such as mannitol, 1,3-butanediol, water, Ringer's solution, or isotonic sodium chloride solution, or suitable dispersing or wetting and suspending agents, such as sterile, non-irritant, fixed oils, including synthetic mono- or diglycerides, and fatty acids, including oleic acid. One illustrative example of a carrier for intravenous use includes a mixture of 10% USP ethanol, 40% USP propylene glycol or polyethylene glycol 600 and the balance USP Water for Injection (WFI). Other illustrative carriers for intravenous use include 10% USP ethanol and USP WFI; 0.01-0.1% triethanolamine in USP WFI; or 0.01-0.2% dipalmitoyl diphosphatidylcholine in USP WFI; and 1-10% squalene or parenteral vegetable oil-in-water emulsion. Water or saline solutions and aqueous dextrose and glycerol solutions may be preferably employed as carriers, particularly for injectable solutions. Illustrative examples of carriers for subcutaneous or intramuscular use include phosphate buffered saline (PBS) solution, 5% dextrose in WFI and 0.01-0.1% triethanolamine in 5% dextrose or 0.9% sodium chloride in USP WFI, or a 1 to 2 or 1 to 4 mixture of 10% USP ethanol, 40% propylene glycol and the balance an acceptable isotonic solution such as 5% dextrose or 0.9% sodium chloride; or 0.01-0.2% dipalmitoyl diphosphatidylcholine in USP WFI and 1 to 10% squalene or parenteral vegetable oil-in-water emulsions.

In one embodiment, the compositions of the subject invention can be formulated for administration via topical application onto the skin, for example, as topical compositions, which include rinse, spray, or drop, lotion, gel, ointment, cream, foam, powder, solid, sponge, tape, vapor, paste, tincture, or using a transdermal patch. Suitable formulations of topical applications can comprise in addition to any of the pharmaceutically active carriers, for example, emollients such as carnauba wax, cetyl alcohol, cetyl ester wax, emulsifying wax, hydrous lanolin, lanolin, lanolin alcohols, microcrystalline wax, paraffin, petrolatum, polyethylene glycol, stearic acid, stearyl alcohol, white beeswax, or yellow beeswax. Additionally, the compositions may contain humectants such as glycerin, propylene glycol, polyethylene glycol, sorbitol solution, and 1,2,6 hexanetriol or permeation enhancers such as ethanol, isopropyl alcohol, or oleic acid.

### Materials and Methods

### Immunogenicity Test

In this test, mice were primed and boosted with the candidate neoantigen vaccines at day 0 and 7. Five days post boost vaccination, mice were sacrificed and splenocytes were harvested for IFN-γ ELISpot assay and flow cytometry.

### Therapeutic Efficacy Test

In this test, mice were transplanted with a subcutaneous model 2×10⁵ MB49 cancer cells on day −8, the multi-epitope neoantigen vaccine was immunized on day 0, 3 and 7, and an additional boost was administered on day 14. The tumor size for each mouse was measured every three days until the humane endpoint. Three days post boost vaccination, mice were sacrificed and splenocytes were harvested for IFN-γ ELISpot assay and flow cytometry.

### IFN-γ ELISpot Assay

After harvesting splenocytes from sacrificed mice, cells were cultured over night at 37° C. on multiscreen filter plates pre-coated with anti-IFN-γ antibody. Murine splenocytes were stimulated by co-incubation with peptides. All samples were tested in triplicate together with the assay positive controls (Staphylococcus enterotoxin B) and cells from a reference donor. All wells were imaged using CTL ImmunoSpot ELISpot Analyzer and analyzed by ImmunoCapture software (Cellular Technology Ltd., Shaker Heights, OH). The immunogenicity of neoantigens was statistically analyzed by Student's t-test and compared with wild type antigens or negative controls.

### Flow Cytometry

After harvesting splenocytes from sacrificed mice, cells were counted and plated at a density of ˜1×10⁶ cells per well in 6-well plates in RPMI medium with 10% FBS and cultured at 37° C. in 5% CO₂ overnight. Cells in each well were stimulated using the penta-peptide mixtures and Brefeldin A (Biolegend, San Diego, CA) was added after 8 hours before harvesting cells for flow cytometry analysis. Brefeldin A (BFA) is a protein transport inhibitor that blocks transport processes during cell activation and is used to enhance intracellular cytokine staining signals. Cells were harvested and then stained for cell surface markers using anti-CD3-FITC, anti-CD4-APC, and anti-CD8-PE. For staining intracellular cytokines (IFNγ), cells were fixed and permeabilized (BD Cytofix/Cytoperm™, Franklin Lakes, NJ), and then stained for IFNγ using anti-IFNγ-PerCP-Cy5.5. Flow cytometry analysis was carried out on a CytoFLEX S flow cytometer (Beckman, Brea, CA). Data processing was performed on FlowJo software.

All patents, patent applications, provisional applications, and publications referred to or cited herein are incorporated by reference in their entirety, including all figures and tables, to the extent they are not inconsistent with the explicit teachings of this specification.

Following are examples that illustrate procedures for practicing the invention. These examples should not be construed as limiting. All percentages are by weight and all solvent mixture proportions are by volume unless otherwise noted.

### Example 1-1V209 Conjugated Peptide Structure

Based on a known antigen peptide, we chemically re-programmed the sequence to convert the antigen into a self-adjuvanting vaccine format, by conjugating antigen motifs to 1V209. 1V209, chemically named 4-[[6-Amino-7,8-dihydro-2-(2-methoxyethoxy)-8-oxo-9H-purin-9-yl]methyl]-benzoic acid with the CAS Number of 1062444-54-5, is a type of synthetic TLR7 agonists with the formula shown in FIG. 1, compound A. In our study, an antigen, an antigenic polypeptide tailed with five lysine (FIG. 1, compound B), is conjugated with five 1V209 molecules by forming amide bonds (blue part in FIG. 1, compound C) between amino groups of the antigen (blue part FIG. 1, compound B) and carboxyl groups of 1V209 (blue part in FIG. 1, compound A). Here, an antigen is a polypeptide, which can induce immune responses, like T cell or B cell responses, after administering into human bodies or animal models. In our study, we tested newly generated antigens, or namely neoantigens in tumor cells because of single nucleotide variations (SNVs), insertions/deletions (Indels), or other types of DNA variations, which changed the coding sequences of normal genes.

### Example 2 Immunogenicity Test of Peptide-Based Self-Adjuvanting Vaccine

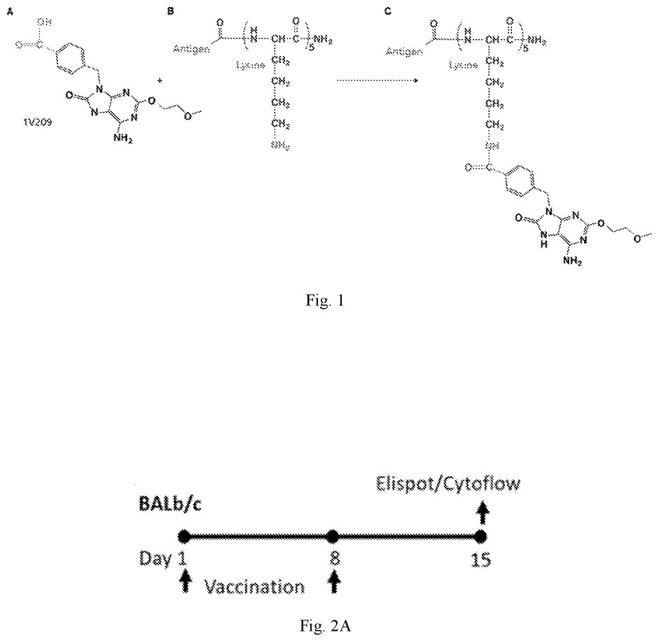

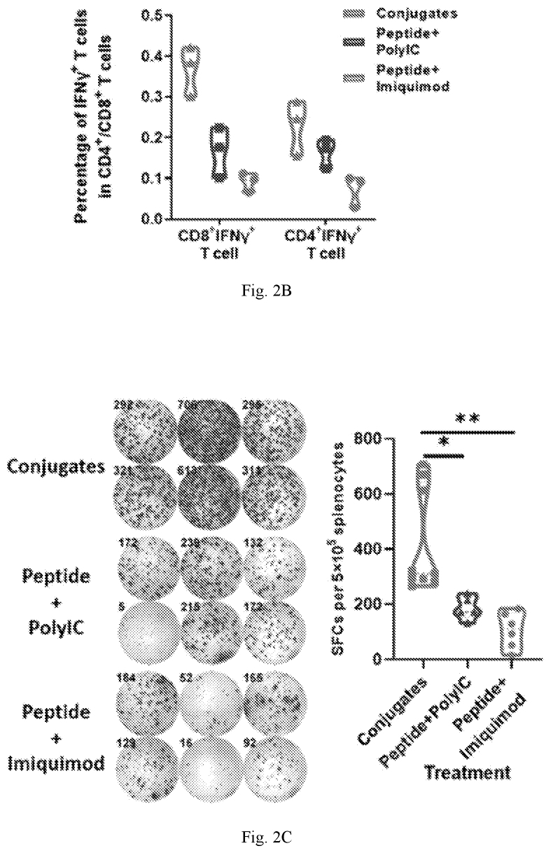

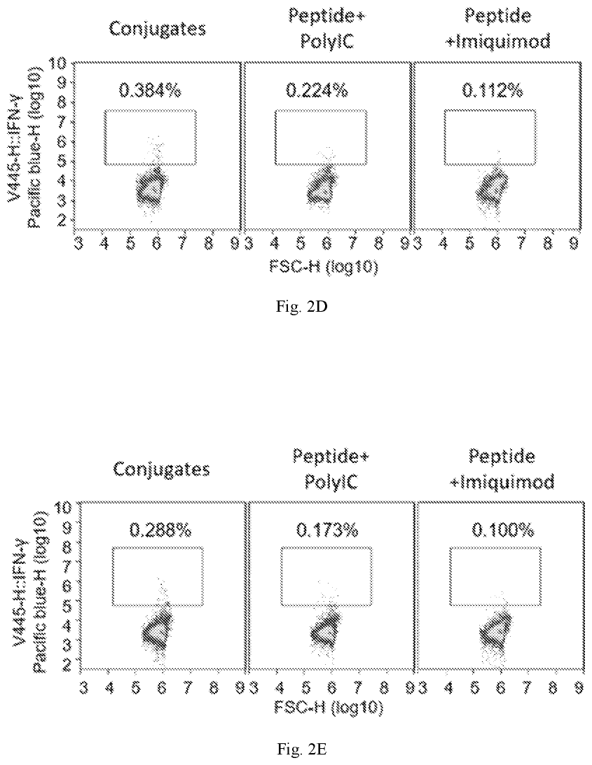

We firstly tested the immunogenicity of 1V209-conjugated peptides in the mouse model (FIG. 2A). By using both IFN-γ ELISpot assay (FIG. 2B) and flow cytometry (FIG. 2C-E), we compared the immunogenicity of poly(I:C) or imiquimod-adjuvanting original peptide with that of our 1V209-conjugated peptide. The peptide we used is a neoantigen from ML-1 murine liver cancer cell line, which a newly generated peptide with A71P alteration in the coding sequence of gene Natdl. Here, poly(I:C) is polyinosinic:polycytidylic acid with the CAS Number of 24939-03-5, which is a type of agonist stimulating TLR3/7/8 pathways as well as RIG-I/MDA5. Imiquimod, chemically named 1-isobutyl-1H-imidazo, quinolin-4-amine with CAS number of 99011-02-6, is a TLR 7/8 agonist. In terms of spot-forming counts in IFN-γ ELISpot assay and percentage of percentage of IFN-γ⁺ T cells in CD8⁺ or CD4⁺ T cells, we illustrated that the 1V209-conjugated peptide induced a stronger T cell response compared with that of the original one at the same dose molar amount (11 nmol).

### Example 3 Peptide-Based Self-Adjuvanting Vaccine in Cancer

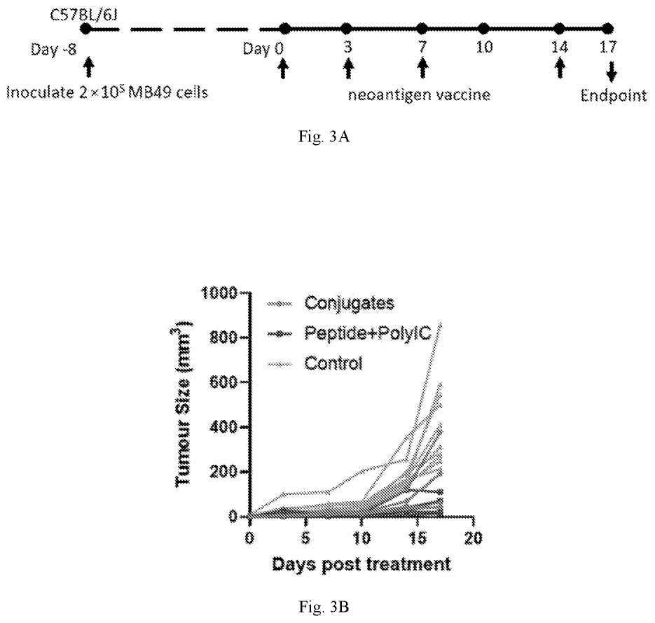

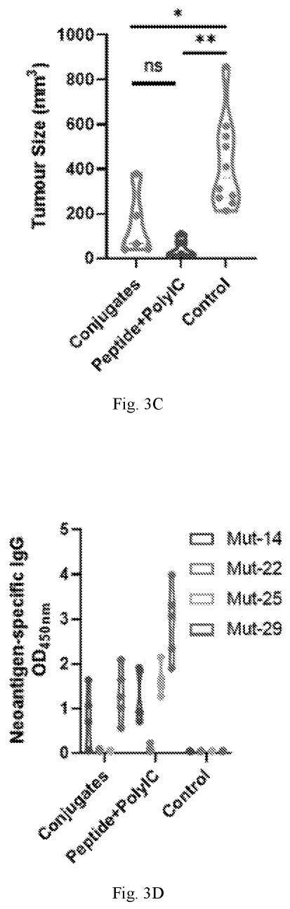

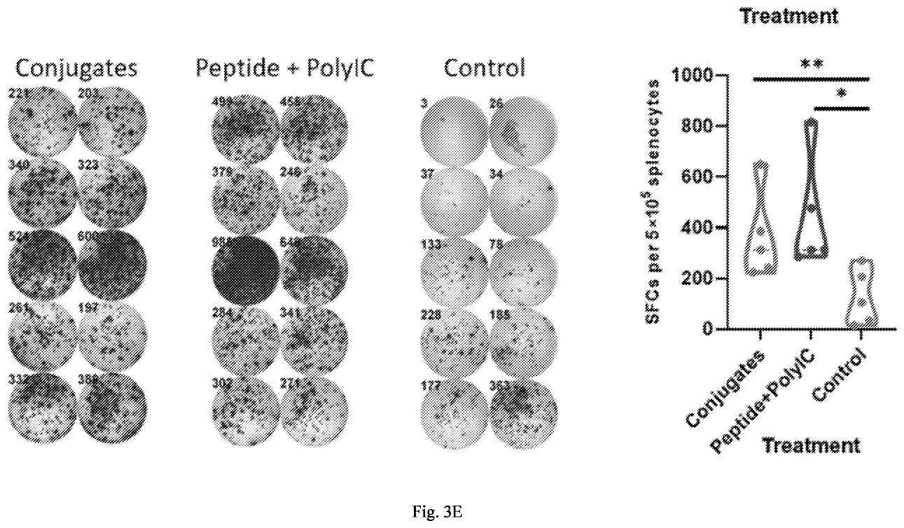

Treatment we further tested the therapeutic efficacy of conjugates, comparing with standard poly(I:C)-adjuvanting peptide. After subcutaneously inoculating 2×10⁵ MB49 bladder cancer cells, we treated the C57BL/6 mice with neoantigen vaccine in the form of 1V209-conjugated peptide or poly(I:C)-adjuvanting peptide at Day 0, 3, 7, and 14 (FIG. 3A). We traced the kinetics of tumor growth in different groups every three days (FIG. 3B). At Day 17, both vaccination groups, 1V209-conjugated peptide (100 μg) or poly(I:C)-adjuvanting original peptide (100 μg peptide+50 μg adjuvant), have shown comparable suppression of tumor growth, comparing with the control groups (FIG. 3C). By IFN-γ ELISpot assay, we found that conjugates almost induced equivalent T cell responses compared with poly(I:C)-adjuvanting original peptide (FIG. 3E). We also detected neoantigen-specific IgG in mouse sera by ELISA at 1:50 dilution. Peptides Mut 14 and Mut 29 induced specific IgG in the 1V209-conjugated peptide group, while peptides Mut14, Mut25, and Mut 29 induced IgG in the poly(I:C)-adjuvanting original peptide group (FIG. 3D). Since all these peptides have been validated with specific T cell response previously (FIG. 3D), some peptides like Mut14, Mut25, and Mut 29 can be considered as chimeric peptides containing both B and T cell epitopes.

It should be understood that the examples and embodiments described herein are for illustrative purposes only and that various modifications or changes in light thereof will be suggested to persons skilled in the art and are to be included within the spirit and purview of this application and the scope of the appended claims. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

### REFERENCES

[1]Ott, P. A., Hu-Lieskovan, S., Chmielowski, B., Govindan, R., Naing, A., Bhardwaj, N., . . . & Srinivasan, L. (2020). A phase Ib trial of personalized neoantigen therapy plus anti-PD-1 in patients with advanced melanoma, non-small cell lung cancer, or bladder cancer. Cell, 183(2), 347-362.
[2]Adams, S., O'Neill, D. W., Nonaka, D., Hardin, E., Chiriboga, L., Siu, K., . . . & Bhardwaj, N. (2008). Immunization of malignant melanoma patients with full-length NY-ESO-1 protein using TLR7 agonist imiquimod as vaccine adjuvant. The Journal of Immunology, 181(1), 776-784.
[3]Carson, D. A., Cottam, H. B., Hayashi, T., Chan, M., & Wu, C. C. (2013). Uses of phospholipid conjugates of synthetic TLR7 agonists. U.S. patent application Ser. No. 13/695,385.
[4]Huang, C. H., Mendez, N., Wang, J., Hayashi, T., Weeks, J., Echeagaray, O. H., . . . & Gude, N. A. (2021). Conjugation of TLR7 agonist to nanomaterials enhances the agonistic activity. U.S. patent application Ser. No. 16/906,933.
[5]Lynn, G. M., & Ishizuka, A. S. (2020). Peptide-based vaccines, methods of manufacturing, and uses thereof for inducing an immune response. U.S. patent application Ser. No. 16/500,762.

1. A peptide-based vaccine conjugate comprising a Toll-like receptor 7 agonist conjugated to a peptide, wherein the Toll-like receptor 7 agonist is 1V209

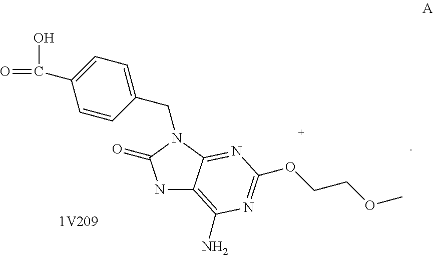 [SMILES: COCCOc1nc(N)c2[nH]c(=O)n(Cc3ccc(C(=O)O)cc3)c2n1]

2. The peptide-based vaccine conjugate of claim 1, wherein the peptide is at least 5, 6, 7, 8, 9, or 10 amino acid residues in length.

3. The peptide-based vaccine conjugate of claim 2, wherein the peptide is 5 amino acid residues in length.

4. The peptide-based vaccine conjugate of claim 3, wherein the peptide comprises a sequence according to SEQ ID NO: 1.

5. The peptide-based vaccine conjugate of claim 3, wherein 1V209 is conjugated to each of the 5 amino acid residues.

6. The peptide-based vaccine conjugate of claim 1, wherein the peptide-based vaccine conjugate is according to formula (I):

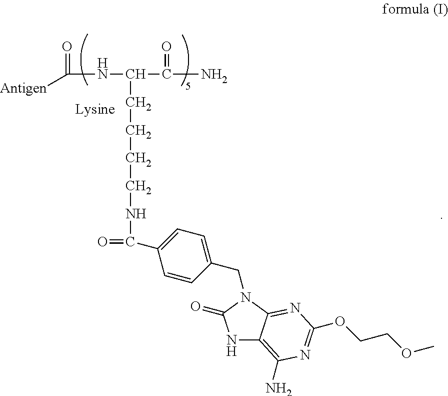

7. The peptide-based vaccine conjugate of claim 1, wherein the peptide-based vaccine conjugate induces an antigen-specific T cell response and antigen-specific B cell response.

8. The peptide-based vaccine conjugate of claim 1, wherein the peptide-based vaccine conjugate inhibits tumor growth.

9. The peptide-based vaccine conjugate of claim 8, wherein the tumor is in the bladder or liver of a subject.

10. A composition comprising a peptide-based vaccine conjugate, wherein the peptide-based vaccine conjugate is according to formula (I):

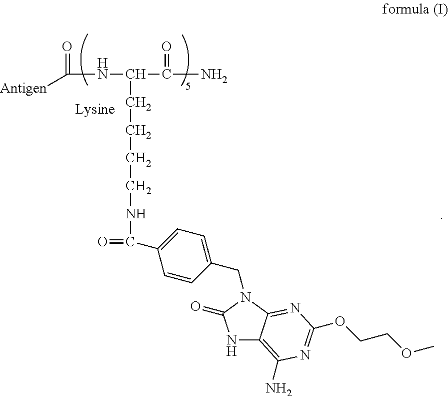

11. The composition of claim 10, further comprising an acceptable carrier and/or excipient.

12. A method of inhibiting tumor growth in a subject, the method comprising administering an effective amount of a peptide-based vaccine conjugate comprising a Toll-like receptor 7 agonist conjugated to a peptide, wherein the Toll-like receptor 7 agonist is 1V209.

13. The method of inhibiting tumor growth of claim 12, wherein the peptide is at least 5, 6, 7, 8, 9, or 10 amino acid residues in length.

14. The method of inhibiting tumor growth of claim 13, wherein the peptide is 5 amino acid residues in length.

15. The method of inhibiting tumor growth of claim 14, wherein the peptide comprises a sequence according to SEQ ID NO: 1.

16. The method of inhibiting tumor growth of claim 14, wherein 1V209 is conjugated to each of the 5 amino acid residues.

17. The method of inhibiting tumor growth of claim 12, wherein the peptide-based vaccine conjugate is according to formula (I):

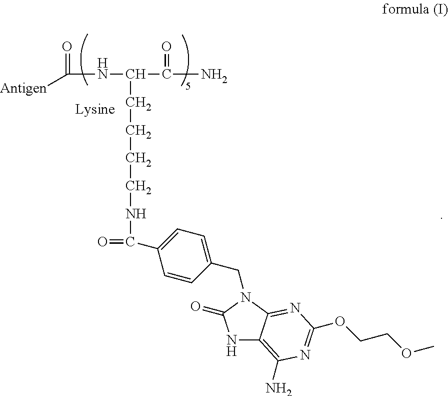

18. The method of inhibiting tumor growth of claim 12, wherein the peptide-based vaccine conjugate induces an antigen-specific T cell response and antigen-specific B cell response.

19. The method of inhibiting tumor growth of claim 18, wherein the antigen-specific T cell responses are CD4+ and CD8+ T cell responses.

20. The method of inhibiting tumor growth of claim 12, wherein the tumor is in the bladder or liver of the subject.
